(19) United States
(12) Patent Application Publication
(10) Pub. No.: US 2014/0094806 A1
(43) Pub. Date: Apr. 3, 2014

(54) SURGICAL INSTRUMENT FOR PENETRATING REMOVING BONE

(71) Applicants: Lanny Leo Johnson, Frankfort, MI (US); Myron Spector, Boston, MA (US)

(72) Inventors: Lanny Leo Johnson, Frankfort, MI (US); Myron Spector, Boston, MA (US)

(21) Appl. No.: 13/633,872

(22) Filed: Oct. 2, 2012

(51) Int. Cl. A61B 17/16

(52) U.S. Cl. 606/79

(57) ABSTRACT

A surgical instrument whose cutting portion is a sharp miniature chisel or awl shape with a series of ridges along the course of the cutting tool. Each ridge has, on its proximal surface, a concave surface. Upon retraction, the instrument is pushed against the side of the defect, allowing the compacted bone on the walls to be captured in the concave recesses of the instrument. Existing instruments compact bone against the walls of the surgical defect rather than removing the compacted bone or tissue. The creation of a small surgically created wound in subchondral bone, absent the thickened compacted bone at the margins, responds with rapid vascular penetration and cellular migration and faster healing with an enhanced histologically confirmed maturing tissue. The surgical defect can be round or slit-like. The instrument allows a wide spectrum of angle of attack to create the surgical defect in the bone and/or articular surface.

### BACKGROUND OF THE INVENTION

The present invention relates to surgical instruments and, more particularly, to a surgical instrument for penetrating bone or an articular joint surface such a manner that, upon removal of the instrument, bone and or cartilage remnants are removed from the sides of the defect so as to avoid leaving compacted bone in the miniature precise defect.

Existing surgical instruments used for penetration into subchondral bone often results in a non-precise defect of relatively large size, pushing bone to the side, resulting in a thickened compacted bony wall that resists or delays vascular penetration into the defect and resulting healing.

Existing awl-type instruments typically used for this purpose are round, tapered with straight, and 45 degree angle, slanted configuration. They have smooth sides that, when penetrating bone, push bone or cartilage to the side of the defect resulting in the bone being compacted to the side and against the walls of the surgical defect.

The existing instruments' geometry allows only a circular awl configuration. Therefore they create large defects with compacted bone on the walls. They do not remove bone from the walls when removed from the defect and, because of their geometry, they cannot create a linear defect and can only have an angle of attack or approach to a bony or articular surface at zero to perhaps 35 (+/−) degrees. When the latter is used, the result can be a much larger defect since the instrument “walks” or moves forward upon impaction.

As can be seen, there is a need for an improved surgical instrument for penetrating bone such that, upon removal, the instrument brings out bone and or cartilage with it to avoid leaving a barrier of compacted bone or tissue in the defect, thus creating a bony defect environment providing for good vascular penetration into the defect and resultant enhanced healing.

### SUMMARY OF THE INVENTION

In one aspect of the present invention, a surgical instrument comprises a main shaft; a plurality of ridges cut along the main shaft, the ridges including a concave surface and concave surface sharp edges; and a sharp tip disposed on a distal end of the main shaft.

In another aspect of the present invention, a method for penetrating bone in a surgical procedure comprises inserting a surgical instrument into bone or an articular joint surface to create a defect, the surgical instrument including a main shaft, a plurality of ridges cut along the main shaft, the ridges including a concave surface and concave surface sharp edges, and a sharp tip disposed on a distal end of the main shaft; and removing the surgical instrument from the defect where the concave surface sharp edges remove compacted bone and tissue from along a sides of the defect and the concave surface collects the compacted bone as the surgical instrument is removed.

These and other features, aspects and advantages of the present invention will become better understood with reference to the following drawings, description and claims.

### BRIEF DESCRIPTION OF THE DRAWINGS

FIG. 1 is a perspective view of a bone penetrating surgical instrument according to an exemplary embodiment of the present invention;

FIG. 2 is a perspective view of the bone penetrating surgical instrument of FIG. 1;

FIG. 3 is a cross-sectional view taken along line 3-3 of FIG. 2;

FIG. 4 is a perspective view of a bone penetrating surgical instrument according to another exemplary embodiment of the present invention;

FIG. 5 is a side view of the bone penetrating surgical instrument of FIG. 4; and

FIG. 6 is a front view of the bone penetrating surgical instrument of FIG. 4.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a surgical instrument in a sharp miniature chisel or awl shape that has a series of ridges along the course of the cutting tool. Each ridge has, on its proximal surface, a concave surface. Upon retraction, the instrument is pushed and or twisted against the side of the defect, allowing the compacted bone on the walls to be captured in the concave recesses of the instrument. Unlike existing instruments that can compact bone against the walls of the surgical defect, the present invention removes the compacted bone from the sides of the surgical defect, improving resultant vascular penetration and healing. The creation of a small surgically created wound in subchondral bone, absent the thickened compacted bone at the margins, responds with rapid vascular penetration/migration and faster healing with an enhanced histologically confirmed maturing tissue. The surgical defect can be round or slit-like. The instrument allows a wide spectrum of angle of attack to create the defect. Slit-type defects in subchondral bone typically heal faster with more mature cartilage tissue.

With the awl and chisel geometry, various angles of approaches are possible in the restricted intra-articular surgical spaces. Moreover, the more desirable slit-like defect is possible in most of the surgical exposures. No bone is compacted with the instrument of the present invention, but is, instead, removed retrograde by the concave surfaces surrounding the tool.

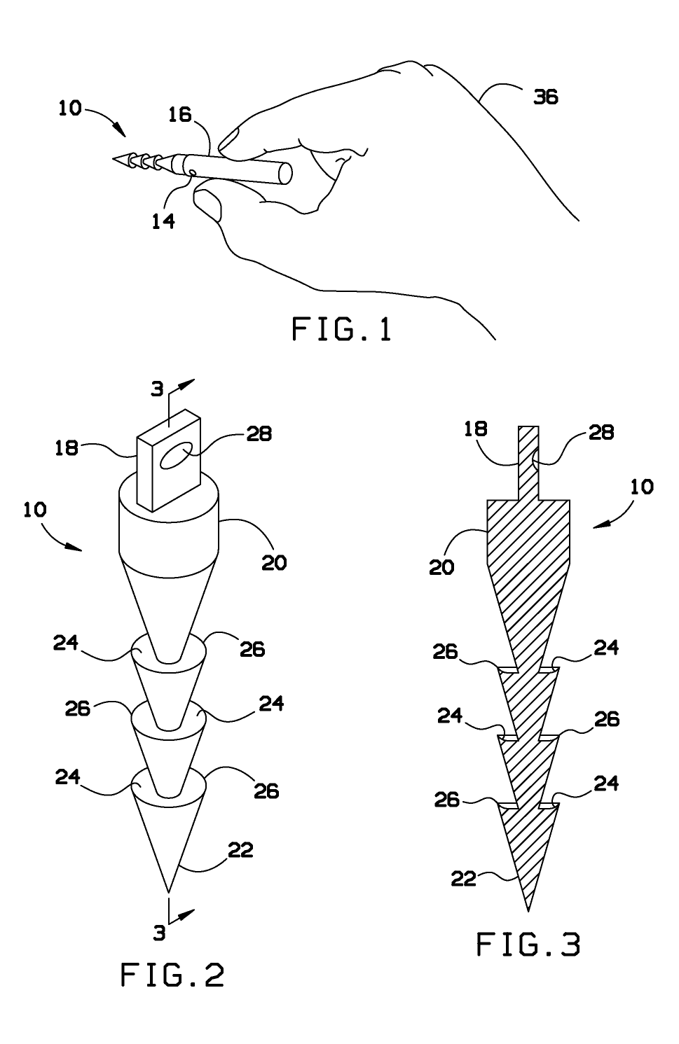

Referring now to FIGS. 1 through 3, an awl-type surgical instrument 10 can be disposed in a hand piece 16, secured with a set screw 14 that can engage with a set screw indention 28 in a hand piece mounting tab 18 of the surgical instrument 10. The hand piece 16 permits a user's hand 36 to hold and control the surgical instrument 10 via the hand piece or handle 16 providing opportunity for use of a hammer or mallet to forcefully create the surgical defect .

An awl main shaft 20 can include a sharp enough tip 22 to initiate penetration into the bone. A series of ridges can be cut along the main shaft 20 between the sharp tip 22 and the hand piece mounting tab 18. Any number of ridges can be disposed along the main shaft 20. Typically from one to five or more ridges may be disposed along the main shaft 20. The ridges can include concave surface sharp edges 26 with concave surfaces 24 formed on each ridge. Each ridge can be formed in a frusto-conical shape. FIG. 3 best shows the concave shape of the concave surfaces 24. The concave surfaces 24 allow the capture of the compacted bone on the walls when the surgical instrument 10 is removed or retracted.

The awl may be conical or angular. The recessed areas to collect bone may be vertical or horizontally oriented depending upon the geometry of the awl surgical instrument. The chisel may have projections on one or two sides for bone collection. Single-sided projections/ridges could be used in small joints to minimize the surgically created defect. In some embodiments, the awl surgical instrument can have ridges that are slanted to vertical. In some embodiments, the chisel surgical instrument can have ridges that are slanted.

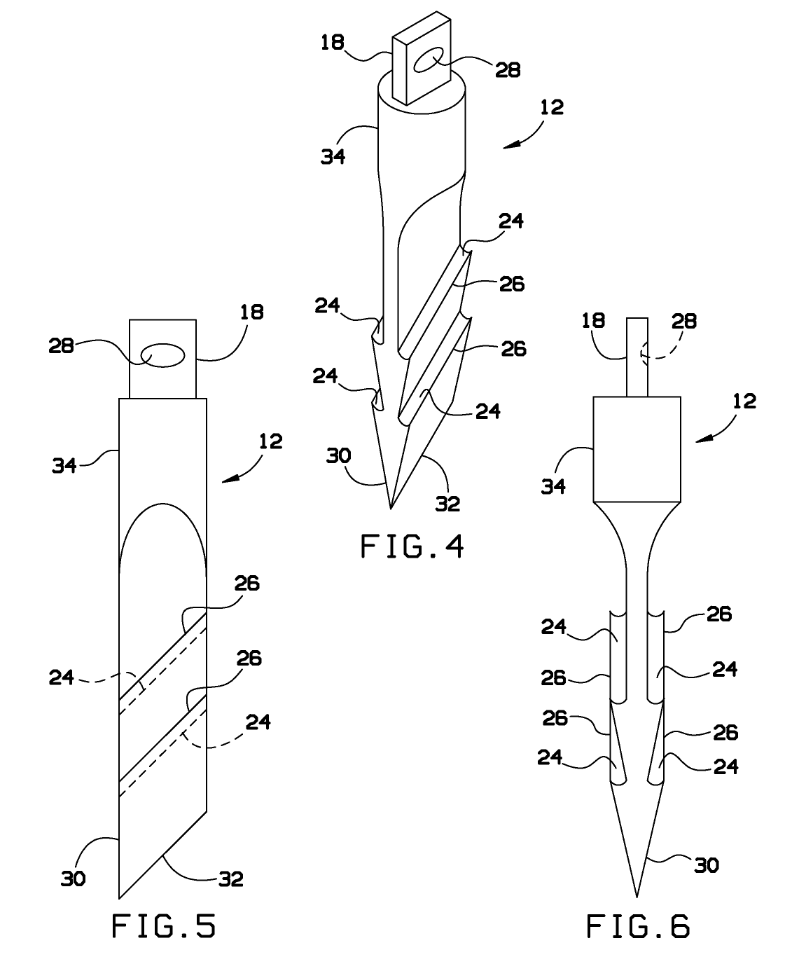

Referring now to FIGS. 4 through 6, a chisel-type surgical instrument 12 can be disposed in the hand piece 16, secured with the set screw or similar secure method of fixation 14 that can engage with the set screw indention 28 in the hand piece mounting tab 18 of the surgical instrument 12, similar to the awl-type surgical instrument 10 described above.. The hand piece 16 permits the user's hand 36 to hold and control the surgical instrument 12 via the hand piece 16 including being of such a configuration to provide for impaction with a mallet or hammer.

A chisel main shaft 34 can include a chisel sharp tip 32. A series of ridges can be cut along the main shaft 34 between the chisel sharp tip 32 and the hand piece mounting tab 18. Any number of ridges can be disposed along the main shaft 34. Typically from one to five or more ridges may be disposed along the main shaft 34. The ridges can include concave surface sharp edges 26 with concave surfaces 24 formed on each ridge. As shown in FIG. 5, the concave surface sharp edges 26 can be disposed at an angle offset from perpendicular from a side edge of the surgical instrument 12. FIG. 6 best shows the concave shape of the concave surfaces 24. The concave surfaces 24 allow the capture of the compacted bone on the walls when the surgical instrument 12 is removed or retracted.

The surgical instruments 10, 12 of the present invention can be made of various materials by various processes, such as by a computer numerical controlled (CNC) machining process. They may be constructed in such a manner that the hand piece is contiguous with the cutting instrument.

The connecting hand piece material may be stainless steel, radel and phenolic. The connection, if not contiguous, is such that rotation is controlled and there is no loosening from the cutting tool upon removal, retraction, or manipulation. This connecting hand piece may be such that provision is made for hammering in and or out.

While the above describes hand tools, using the hand piece 16, the surgical instruments 10, 12 of the present invention could be mechanized, motor driven or other energy sources, is possible or feasible, depending on the application.

It should be understood, of course, that the foregoing relates to exemplary embodiments of the invention and that modifications may be made without departing from the spirit and scope of the invention as set forth in the following claims.

What is claimed is:

1. A surgical instrument comprising:
a main shaft attached to a handle;
a plurality of ridges cut along the main shaft, the ridges including a concave surface and concave surface sharp edges; and
a sharp tip disposed on a distal cutting end of the main shaft.

2. The surgical instrument of claim 1, wherein the main shaft is tapered at the tip.

3. The surgical instrument of claim 1, wherein the main shaft is a chisel type configured tip.

4. The surgical instrument of claim 3, wherein the concave surface sharp edges are disposed at an angle offset from perpendicular from a side edge of the surgical instrument.

5. The surgical instrument of claim 1, further comprising a hand piece contiguous with a proximal end of the main shaft

6. The plurality of projecting surfaces are horizontal in spatial relationship to the axis of the shaft, comprised of a proximal broad concave surface bordered by sharp cutting edges for the removal and collection of bone from the sides of the bore.

7. (canceled)

8. A method for penetrating bone or an articular joint surface in a surgical procedure, the method comprising:
inserting a surgical instrument into bone or an articular surface to create a defect, the surgical instrument including a main shaft, a plurality of ridges cut along the main shaft, each of the plurality of ridges including a concave surface and concave surface sharp edges, and a sharp tip disposed on a distal end of the main shaft; and
removing the surgical instrument from the defect where the horizontal concave surface sharp edges remove compacted bone from along a surface of the defect

9. The method of claim 8, wherein the sharp cutting tip disposed on the distal end of the main shaft is tapered.

10. The surgical instrument of claim 8, wherein the main shaft is a chisel main shaft having a rectangular cross section and the broad horizontal projection has a concave surface that collects the compacted bone as the surgical instrument is removed.

10. The surgical instrument of claim 8, wherein the main shaft is a chisel type geometry with a shaft having a rectangular cross-section.

11. The surgical instrument of claim 10, wherein the broad horizontal concave surface has sharp edges that are disposed at an angle offset from perpendicular from a side edge of the surgical instrument.
